SEQUENCE LISTING (3 sequences)

SEQ ID NO: 1 - DNA, 1394 bp, Bifidobacterium animalis
  source 1..1394: mol_type = genomic DNA; sub_species = lactis
caagggtcgggccaccggcttcgggtgctacccactttcatgacttgacgggcggtgtgtacaaggcccgggaacgcattcaccgcggcgttgctgatccgcgattactagcgactccgccttcacgcagtcgagttgcagactgcgatccgaactgagaccggttttcagcgatccgccccacgtcaccgtgtcgcaccgcgttgtaccggccattgtagcatgcgtgaagccctggacgtaaggggcatgatgatctgacgtcatccccaccttcctccgagttgaccccggcggtcccacatgagttcccggcatcacccgctggcaacatgcggcgagggttgcgctcgttgcgggacttaacccaacatctcacgacacgagctgacgacgaccatgcaccacctgtgaaccggccccgaagggaaaccgtgtctccacggcgatccggcacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttggctccgacacgggacccgtggaaagggccccacatccagcatccaccgtttacggcgtggactaccagggtatctaatcctgttcgctccccacgctttcgctcctcagcgtcagtgacggcccagagacctgccttcgccattggtgttcttcccgatatctacacattccaccgttacaccgggaattccagtctcccctaccgcactccagcccgcccgtacccggcgcagatccaccgttaggcgatggactttcacaccggacgcgacgaaccgcctacgagccctttacgcccaataaatccggataacgctcgcaccctacgtattaccgcggctgctggcacgtagttagccggtgcttattcgaacaatccactcaacacggccgaaaccgtgccttgcccttgaacaaaagcggtttacaacccgaaggcctccatcccgcacgcggcgtcgctgcatcaggcttgcgcccattgtgcaatattccccactgctgcctcccgtaggagtctgggccgtatctcagtcccaatgtggccggtcaccctctcaggccggctacccgtcaacgccttggtgggccatcaccccgccaacaagctgataggacgcgaccccatcccatgccgcaaaagcatttcccaccccaccatgcgatggagcggagcatccggtattaccacccgtttccaggagctattccggtgcacagggcaggttggtcacgcattactcacccgttcgccactctcaccccgacagcaagctgccagggatcccgttcgactgcatg

SEQ ID NO: 2 - DNA, 17 bp, synthetic construct
  source 1..17: mol_type = other DNA
  misc_difference 9: note = a, c, g, or t
cctacgggnggcwgcag

SEQ ID NO: 3 - DNA, 21 bp, synthetic construct
  source 1..21: mol_type = other DNA
gactachvgggtatctaatcc

(19) United States
(12) Patent Application Publication
(10) Pub. No.: US 2024/0287447 A1
(43) Pub. Date: Aug. 29, 2024

(54) BIFIDOBACTERIUM ANIMALIS SUBSP. LACTIS BLA36 FOR RELIEVING CONSTIPATION, METHOD FOR CULTURING SAME, AND USE THEREOF

(71) Applicant: WECARE PROBIOTICS CO., LTD., Suzhou, Jiangsu (CN)

(72) Inventors: Shuguang FANG, Suzhou, Jiangsu (CN); Yao DONG, Suzhou, Jiangsu (CN); Zhonghui GAI, Suzhou, Jiangsu (CN); Jiayue GU, Suzhou, Jiangsu (CN); Junli ZHANG, Suzhou, Jiangsu (CN); Jianguo ZHU, Suzhou, Jiangsu (CN)

(73) Assignee: WECARE PROBIOTICS CO., LTD., Suzhou, Jiangsu (CN)

(21) Appl. No.: 18/569,535

(22) PCT Filed: Jul. 18, 2022
(86) PCT No.: PCT/CN2022/106181

§ 371 Date: Dec. 12, 2023

(30) Foreign Application Priority Data
Mar. 17, 2022 (CN) 202210261807.5

(51) Int. Cl. C12N 1/20; A61K 35/745; A61P 1/10; C12R 1/01

(52) U.S. Cl. CPC C12N 1/205; A61K 35/745; A61P 1/10; C12R 2001/01

(57) ABSTRACT

The present invention provides a Bifidobacterium animalis subsp. lactis strain BLa36 for relieving constipation, a method for culturing same, and use thereof. The Bifidobacterium animalis subsp. lactis strain BLa36 for relieving constipation is designated as Bifidobacterium animalis subsp. lactis BLa36, which has been deposited at the China General Microbiological Culture Collection Center on Dec. 2, 2021, with an accession number of CGMCC No. 24029, and the address for the deposition is Yard No. 1(3), West Beichen Road, Chaoyang District, Beijing, China. The Bifidobacterium animalis subsp. lactis has good resistance to stomach acid and can relieve constipation.

### TECHNICAL FIELD

The present application belongs to the technical field of microbial culture, and relates to Bifidobacterium animalis subsp. lactis BLa36 for relieving constipation, a method for culturing the same and use thereof.

### BACKGROUND

Intractable constipation is a common syndrome. In recent years, the incidence of constipation has been increasing gradually as the society changes in habits and diet. Epidemiologic surveys have shown that the prevalence of constipation ranges from 2% to 27% in various countries.

After excluding the constipation caused by organic and metabolic diseases, chronic constipation can be divided into three subtypes: slow transit constipation (STC), outlet obstruction constipation, and mixed constipation, wherein the STC accounts for 45.5% of chronic constipation. The conception of STC was originally proposed by Proton and Lennar-Joners in 1986, and after three decades of research, it is now generally accepted that STC is a type of intractable functional constipation characterized by slow fecal transport in the intestinal tract, which is most common in the elderly and pregnancy women. However, since the pathogenesis of STC has not been fully clarified, the conservative medical treatment only has poor long-term therapeutic effect and cannot solve the problem fundamentally, and thus surgical intervention is the last resort after the failure of non-surgical treatment for STC.

Slow transit constipation (STC) is a common chronic intractable constipation in clinical practice, manifested as intestinal motility disorders and delayed transmission of intestinal contents caused by complex factors, and has the characteristics of chronic, primary, and functional. The etiology of STC is not fully understood, and its clinical manifestations mainly include low stool frequency, no intestinal tract movement sensation, no dragging sensation and no defecating desire, and there are symptoms of abdominal distension and food intake inability due to the prolonged absence of evacuation of the bowels. Slow transit constipation is a chronic intestinal disease that is difficult to treat. In the past, the treatment of slow transit constipation was mainly based on conservative medical treatment. However, the conservative treatment can only temporarily relieve the symptoms, but cannot fully fix the constipation, or even cause more severe symptoms or other complications, and the long-term therapeutic effect is not satisfactory. Slow transit constipation is a common health problem usually attributed to intestine motility disorders, such as decreased colonic propulsive function, overall reduction in colonic electrical or motility activity, and altered rectosigmoid contractile activity, and abnormal response to food intake. Constipation has a high prevalence among various gastrointestinal disorders.

Constipation is currently treated with medications that can increase fecal moisture content and gastrointestinal vitality factors. However, these drugs have side effects. Studies have shown that constipation is closely related to intestinal microorganisms, and the intestinal microorganisms can be used as an alternative method for constipation intervention and treatment. For example, CN111603489A discloses a probiotic preparation for relieving constipation and a preparation method thereof, comprising a Bacillus coagulans BC99 powder and excipients; the Bacillus coagulans strain BC99 is safe and non-toxic, and effective in improving defecation and fecal characteristics in constipated patients; the purpose of promoting intestinal health is achieved.

CN109897795A discloses a microbial probiotic preparation and use in the prevention and treatment of constipation; the probiotic preparation is Bacillus amyloliquefaciens YHSH-58, with a deposit number of CGMCC NO. 13664 and a deposit date of Feb. 16, 2017, which can be used in the prevention and treatment of constipation.

However, microbial preparations for relieving constipation are limited in the prior art so far, and the effectiveness of these preparations for relieving constipation still needs to be further improved.

Therefore, it is a an urgent problem to be solved by those skilled in the art about how to provide a microbial preparation which is effective in relieving or treating constipation and does not cause any adverse reactions in patients during the treatment process.

### SUMMARY

The present application provides Bifidobacterium animalis subsp. lactis BLa36 for relieving constipation, a method for culturing the same, and use thereof.

In a first aspect, the present application provides Bifidobacterium animalis subsp. lactis BLa36 for relieving constipation, named as Bifidobacterium animalis subsp. lactis strain BLa36, which has been deposited in the China General Microbiological Culture Collection Center on Dec. 2, 2021 with a deposit number of CGMCC No. 24029, and a deposit address of No. 3, No. 1 Courtyard, West Beichen Road Chaoyang District Beijing.

In a second aspect, the present application provides a method for culturing the Bifidobacterium animalis subsp. lactis BLa36 for relieving constipation according to the first aspect, which comprises inoculating the Bifidobacterium animalis subsp. lactis BLa36 on an MRS medium for culturing.

Preferably, the culturing is performed at 30-37° C., such as 30° C., 31° C., 32° C., 33° C., 34° C., 35° C., 36° C. or 37° C., etc., and the culturing is performed for 18-24 h, such as 18 h, 19 h, 20 h, 21 h, 22 h, 23 h or 24 h, etc.

In a third aspect, the present application provides a probiotic preparation for relieving constipation, which comprises the Bifidobacterium animalis subsp. lactis BLa36 for relieving constipation according to the first aspect and a protective agent.

Preferably, a mass ratio of the protective agent to the Bifidobacterium animalis subsp. lactis is (1.8-2.3):1;

wherein specific values in the (1.8-2.3) are, for example, 1.8, 1.9, 2, 2.1, 2.2 or 2.3, etc.

Preferably, the protective agent comprises a skimmed milk powder.

Preferably, the protective agent comprises a skimmed milk powder solution, and the skimmed milk powder solution has a mass concentration of 8%-12%, such as 8%, 9%, 10%, 11% or 12%, etc.

Preferably, the probiotic preparation for relieving constipation further comprises a prebiotic, and the prebiotic comprises any one or a combination of at least two of oligofructose, oligogalactose, xylooligosaccharide, isomaltooligosaccharide, soybean oligosaccharide, inulin, spirulina, arthrospira, Coriolus versicolor polysaccharide, stachyose, polydextrose, α-lactalbumin or lactoferrin; wherein the combination of at least two is, for example, a combination of inulin and spirulina, a combination of stachyose and polydextrose, or a combination of Coriolus versicolor polysaccharide and stachyose, or any other combinations.

Preferably, the probiotic preparation for relieving constipation further comprises Bifidobacterium animalis subsp. lactis BLa80, named as Bifidobacterium animalis subsp. lactis strain BLa80, which has been deposited in the China General Microbiological Culture Collection Center on Mar. 5, 2018 with a deposit number of CGMCC NO. 15410, and a deposit address of No. 3, No. 1 Courtyard, West Beichen Road Chaoyang District Beijing.

Preferably, a mass ratio of the Bifidobacterium animalis subsp. lactis BLa36 to the Bifidobacterium animalis subsp. lactis BLa80 is (1-3):(1-3);

wherein specific values in the (1-3) are, for example, 1, 1.2, 1.5, 1.8, 2, 2.2, 2.5, 2.8 or 3, etc.

In a fourth aspect, the present application provides use of the Bifidobacterium animalis subsp. lactis BLa36 for relieving constipation according to the first aspect or the probiotic preparation for relieving constipation according to the third aspect in the preparation of a product for relieving or treating constipation, wherein the product comprises a foodstuff, a nutraceutical or a drug.

Preferably, in the product for relieving or treating constipation, the Bifidobacterium animalis subsp. lactis BLa36 has a viable count of not less than 1×10⁸ CFU/mL.

Preferably, in the drug, the Bifidobacterium animalis subsp. lactis BLa36 has a viable count of not less than 1×10¹⁰ CFU/mL.

The numerical ranges in the present application comprise not only the point values listed above, but also any unlisted point values within the numerical ranges. Due to the concerns for space and brevity, the specific point values included in the numerical ranges will not be listed exhaustively in present application.

Compared with the prior art, the present application has the following beneficial effects.

In the present application, the Bifidobacterium animalis subsp. lactis strain BLa36 is screened out, which has good gastric acid resistance and relieves slow transport constipation, reflected as the increased fecal moisture content, increased intestinal transit rate, decreased nitric oxide (NO), nitric oxide synthase (NOS) and vasoactive intestinal peptide (VIP) in serum, and increased marker c-kit of interstitial cells of Cajal (ICC) at protein and mRNA levels. In addition, the Bifidobacterium animalis subsp. lactis BLa36 can regulate the intestinal flora, increase the α-diversity of the intestinal flora, and increase the relative abundance of beneficial bacteria. The Bifidobacterium animalis subsp. lactis BLa36 will not lead to drug resistance when used for treatment, and does not cause adverse reactions in patients during the treatment, and can be used for the preparation of products for relieving or treating constipation, such as drugs, foodstuffs or nutraceuticals, with a promising application prospect.

Compared with the Bifidobacterium animalis disclosed in the prior art, the Bifidobacterium animalis subsp. lactis BLa36 provided in the present application has a significantly better effect in relieving constipation. In addition, it is also unexpectedly found in the present application that the constipation-relieving effect of the composite probiotic preparation obtained by compounding BLa36 with BLa80 is improved relative to BLa36 used alone. The two bacterial strains may promote each other's growth and thus have a synergistic effect in relieving constipation.

### BRIEF DESCRIPTION OF DRAWINGS

FIG. 1 shows the effect of Bifidobacterium animalis subsp. lactis BLa36 on the moisture content of rat feces.

FIG. 2 shows the effect of Bifidobacterium animalis subsp. lactis BLa36 on the intestinal propulsion rate of rats.

FIG. 3 shows the intervention of Bifidobacterium animalis subsp. lactis BLa36 on the vasoactive intestinal peptide (VIP) in rat serum.

FIG. 4 shows the intervention of Bifidobacterium animalis subsp. lactis BLa36 on the concentration of substance P in serum.

FIG. 5 shows the intervention of Bifidobacterium animalis subsp. lactis BLa36 on the concentration of nitric oxide synthase (NOS) in serum.

FIG. 6 shows the intervention of Bifidobacterium animalis subsp. lactis BLa36 on the concentration of nitric oxide (NO) in serum.

FIG. 7 shows the effect of Bifidobacterium animalis subsp. lactis BLa36 on the expression level of c-kit in the intestinal mucosa of constipated rats.

FIG. 8 shows the effect of Bifidobacterium animalis subsp. lactis BLa36 on the α-diversity of intestinal microbial community in rats.

FIG. 9 shows the effect of Bifidobacterium animalis subsp. lactis BLa36 on the differential flora of intestinal microbial community in rats.

FIG. 10 shows the change in intestinal propulsion rate after the rats are treated with nutraceuticals containing Bifidobacterium animalis subsp. lactis BLa36.

### DETAILED DESCRIPTION

The technical solutions of the present application are further described below through specific embodiments. It should be apparent to those skilled in the art that the examples are merely used for a better understanding of the present application and should not be regarded as a specific limitation on the present application. Information on the raw materials involved in the specific embodiments is as follows.

MRS solid medium: 10 g of peptone, 10 g of beef paste, 20 g of glucose, 2 g of sodium acetate, 5 g of yeast powder, 2 g of diammonium hydrogen citrate, 2.6 g of K₂PO₄·3H₂O, 0.1 g of MgSO₄ 7H₂O, 0.05 g of MnSO₄, 20 g of agar and 0.5 g of L-cysteine were weighed out, dissolved with deionized water, and added with 1 mL of Polysorbate 80, then deionized water continued to be added until a total volume of the system reached 1 L, and the system was sterilized and cooled, and then poured into a sterilized culture dish for later use.

MRS liquid medium: 10 g of peptone, 10 g of beef paste, 20 g of glucose, 2 g of sodium acetate, 5 g of yeast powder, 2 g of diammonium hydrogen citrate, 2.6 g of K₂PO₄·3H₂O, 0.1 g of MgSO₄·7H₂O, 0.05 g of MnSO₄ and 0.5 g of L-cysteine were weighed out, dissolved with deionized water, and added with 1 mL of Polysorbate 80, then deionized water continued to be added until a total volume of the system reached 1 L, and the system was sterilized and cooled for later use.

### Example 1

This example provides Bifidobacterium animalis subsp. lactis BLa36 for relieving constipation, which is isolated and screened as follows.

A breast milk sample was selected, subjected to ten-fold gradient dilution with normal saline having a mass concentration of 0.9% for 3 times, spread on the MRS solid medium (the medium contains the antibiotic mupirocin lithium salt, which can inhibit most of the strains except bifidobacteria, and the drug is purchased from Hope Bio), and incubated at 37° C. for 48 h, three colonies with different morphologies were picked out and streaked across the surface of the MRS solid medium for isolation, and single colonies were picked out and cultured with the MRS liquid medium at 37° C., and then preserved with glycerol having a mass concentration of 30%. In vitro physiological characterization was carried out on the three preserved human milk-derived single-strains, to screen a single-strain having the best performance in gastrointestinal fluid tolerance (under artificially simulated conditions), HT-29 cell adhesion, bacteriostatic ability and functional oligosaccharide utilization ability.

### Example 2

In this example, the Bifidobacterium animalis subsp. lactis BLa36 screened in Example 1 was subjected to morphological identification and 16S rRNA molecular biological identification, the steps of which are as follows.

### (1) Morphological Identification:

The strain was inoculated in the MRS solid medium and incubated at 37° C. for 48 h and then observed by a microscope.

It can be seen through the observation that the colony is milky white and semi-circular raised, and has smooth, moist surface and neat edge.

(2) 16S rRNA Molecular Biological Identification:

The strain preserved at −80° C. was taken out, inoculated into a centrifuge tube containing 20 mL of MRS liquid medium with a ratio of 2%, incubated at 37° C. for 18 h and then centrifuged at 8000 rpm for 10 min; the supernatant was removed, and the bacterial pellet was collected.

The genome of the strain was extracted and subjected to PCR amplification with bacterial universal primers, and the amplified product was sent to Sangon Biotech (Shanghai) Co., Ltd. for sequencing analysis.

Based on the sequencing analysis, the 16S rDNA sequence of the strain is shown in SEQ ID No: 1.

The sequence obtained from the sequencing analysis was subjected to sequence alignment in GenBank, and the results show that the strain is Bifidobacterium animalis subsp. lactis indeed.

SEQ ID No: 1:
CAAGGGTCGGGCCACCGGCTTCGGGTGCTACCCACTTTCATGACTTGAC
GGGCGGTGTGTACAAGGCCCGGGAACGCATTCACCGCGGCGTTGCTGAT
CCGCGATTACTAGCGACTCCGCCTTCACGCAGTCGAGTTGCAGACTGCG
ATCCGAACTGAGACCGGTTTTCAGCGATCCGCCCCACGTCACCGTGTCG
CACCGCGTTGTACCGGCCATTGTAGCATGCGTGAAGCCCTGGACGTAAG
GGGCATGATGATCTGACGTCATCCCCACCTTCCTCCGAGTTGACCCCGG
CGGTCCCACATGAGTTCCCGGCATCACCCGCTGGCAACATGCGGCGAGG
GTTGCGCTCGTTGCGGGACTTAACCCAACATCTCACGACACGAGCTGAC
GACGACCATGCACCACCTGTGAACCGGCCCCGAAGGGAAACCGTGTCTC
CACGGCGATCCGGCACATGTCAAGCCCAGGTAAGGTTCTTCGCGTTGCA
TCGAATTAATCCGCATGCTCCGCCGCTTGTGCGGGCCCCCGTCAATTTC
TTTGAGTTTTAGCCTTGCGGCCGTACTCCCCAGGCGGGATGCTTAACGC
GTTGGCTCCGACACGGGACCCGTGGAAAGGGCCCCACATCCAGCATCCA
CCGTTTACGGCGTGGACTACCAGGGTATCTAATCCTGTTCGCTCCCCAC
GCTTTCGCTCCTCAGCGTCAGTGACGGCCCAGAGACCTGCCTTCGCCAT
TGGTGTTCTTCCCGATATCTACACATTCCACCGTTACACCGGGAATTCC
AGTCTCCCCTACCGCACTCCAGCCCGCCCGTACCCGGCGCAGATCCACC
GTTAGGCGATGGACTTTCACACCGGACGCGACGAACCGCCTACGAGCCC
TTTACGCCCAATAAATCCGGATAACGCTCGCACCCTACGTATTACCGCG
GCTGCTGGCACGTAGTTAGCCGGTGCTTATTCGAACAATCCACTCAACA
CGGCCGAAACCGTGCCTTGCCCTTGAACAAAAGCGGTTTACAACCCGAA
GGCCTCCATCCCGCACGCGGCGTCGCTGCATCAGGCTTGCGCCCATTGT
GCAATATTCCCCACTGCTGCCTCCCGTAGGAGTCTGGGCCGTATCTCAG
TCCCAATGTGGCCGGTCACCCTCTCAGGCCGGCTACCCGTCAACGCCTT
GGTGGGCCATCACCCCGCCAACAAGCTGATAGGACGCGACCCCATCCCA
TGCCGCAAAAGCATTTCCCACCCCACCATGCGATGGAGCGGAGCATCCG
GTATTACCACCCGTTTCCAGGAGCTATTCCGGTGCACAGGGCAGGTTGG
TCACGCATTACTCACCCGTTCGCCACTCTCACCCCGACAGCAAGCTGCC
AGGGATCCCGTTCGACTGCATG.

### Example 3

In this example, culture conditions of Bifidobacterium animalis subsp. lactis BLa36 were optimized, the steps of which are as follows.

The Bifidobacterium animalis subsp. lactis BLa36 was inoculated into the MRS liquid medium and cultured for 48 h at different temperatures from 10-50° C. separately; during the culture process, the OD₆₀₀ value of the culture solution was measured by an instrument for enzyme-linked immunosorbent assay at set intervals.

The results show that the Bifidobacterium animalis subsp. lactis BLa36 grows best at 30-37° C., and after 18-24 hours of culture, the growth reaches the stationary phase.

### Example 4

In this example, the Bifidobacterium animalis subsp. lactis BLa36 is tested for gastric acid resistance, the steps of which are as follows.

### (1) Preparation of Artificial Gastric Fluid:

The artificial gastric fluid contained 0.20% NaCl and 0.30% pepsin by mass fraction, the pH was separately adjusted to 2.0, 2.5, and 3.0 using HCl, and the artificial gastric fluid was filtered and sterilized for later use.

### (2) Gastric Acid Resistance Test:

The 1.0 mL of Bifidobacterium animalis subsp. lactis BLa36 suspension was taken (with a concentration of 1×10⁹ CFU/mL; the concentration of bacterial suspension is detected by the method in Chinese Standard “GB4789.35-2016: National food safety standard—Microbiological examination of food—Examination of lactic acid bacteria”) (The Bifidobacterium animalis subsp. lactis BLa36 suspension: the Bifidobacterium animalis subsp. lactis was inoculated into the MRS liquid medium, incubated at 37° C. for 18 h for activation, and activated for 2 times consecutively to obtain an activation solution; the activation solution was inoculated into the MRS liquid medium at an inoculum amount of 2% (v/v), and incubated at 37° C. for 18 h to obtain a bacterial solution; the bacterial solution was centrifuged at 8000 g for 10 min, and the supernatant was separated out and filtered with a 0.22 μm sterile filtration membrane to obtain a supernatant of Bifidobacterium animalis subsp. lactis; the bacterial pellet was re-suspended with PBS, so as to obtain the desired bacterial suspension), the Bifidobacterium animalis subsp. lactis BLa36 suspension was mixed with 9.0 mL of artificial gastric fluid at pH 2.0, 2.5 and 3.0, respectively, and incubated at 37° C. anaerobically and statically, and samples were taken at the beginning (0 h) and at the third hour of the treatment, respectively, and the number of viable bacteria was counted by the pour plate method and calculated for the survival rate according to the following formula:

Survival rate (%)=N1/N0×100%,

wherein N1 is the viable count after 3 hours of artificial gastric fluid treatment; NO is the viable count at 0 h. The test results are shown in Table 1.

TABLE 1

| pH of artificial | | | Survival |
|---|---|---|---|
| gastric fluid | Viable count N0 (0 h) | Viable count N1 (3 h) | rate (%) |
| 2.0 | (5.80 ± 0.32) × 10⁸ | (4.47 ± 0.11) × 10⁸ | 77.0 |
| 2.5 | (5.79 ± 0.41) × 10⁸ | (5.31 ± 0.26) × 10⁸ | 91.5 |
| 3.0 | (5.78 ± 0.32) × 10⁸ | (5.43 ± 0.29) × 10⁸ | 93.7 |

As can be seen from Table 1, the Bifidobacterium animalis subsp. lactis BLa36 of the present application has good gastric acid resistance, and the survival rate can reach 77.0% or more after 3 hours of incubation in an artificial gastric fluid at pH 2.0; the survival rate can reach 91.5% or more after 3 hours of incubation in an artificial gastric fluid at pH 2.5; the survival rate can reach 93.7% or more after 3 hours of incubation in an artificial gastric fluid at pH 3.0. The good acid resistance lays the groundwork for preparing BLa36 into products for relieving or treating constipation.

### Example 5

In this example, the Bifidobacterium animalis subsp. lactis BLa36 is tested for the effect in restoring intestinal function, the steps of which are as follows.

(1) Establishment of constipated rat model: rats were administrated with loperamide to induce constipation according to the common method in the prior art. Thirty rats were randomly divided into 3 groups (10 rats in each group): a normal control group (CTL), an untreated slow transport constipation group (STC), and a treated slow transport constipation rat group with Bifidobacterium animalis subsp. lactis BLa36 (BLa36). CTL: normal saline (10 mL/kg) treatment; STC: loperamide (10 mg/kg) treatment; BLa36: loperamide (10 mg/kg)+Bifidobacterium animalis subsp. lactis BLa36 (10⁹ CFU/day) treatment. Loperamide was orally administrated once daily for 14 days to induce constipation in the groups except CTL rats. The Bifidobacterium animalis subsp. lactis BLa36 suspension was suspended in water, and the BLa36 group was administered orally once daily from day 15 to day 28, and the CTL group and STC group were gavaged with normal saline at 1 mL/100 g.

(2) The Bifidobacterium animalis subsp. lactis BLa36 was inoculated into the MRS liquid medium and incubated at 37° C. for 18 h for activation, and activated for 2 times consecutively to obtain an activation solution; the activation solution was inoculated into the MRS liquid medium at an inoculum amount of 2% (v/v), and incubated at 37° C. for 18 h to obtain a bacterial solution; the bacterial solution was centrifuged at 8000 g for 10 min, and the supernatant was separated out and filtered with a 0.22 μm sterile filtration membrane to obtain a supernatant; the bacterial pellet was re-suspended with PBS, so as to obtain the Bifidobacterium animalis subsp. lactis BLa36 suspension.

### (3) Fecal Moisture Content Test:

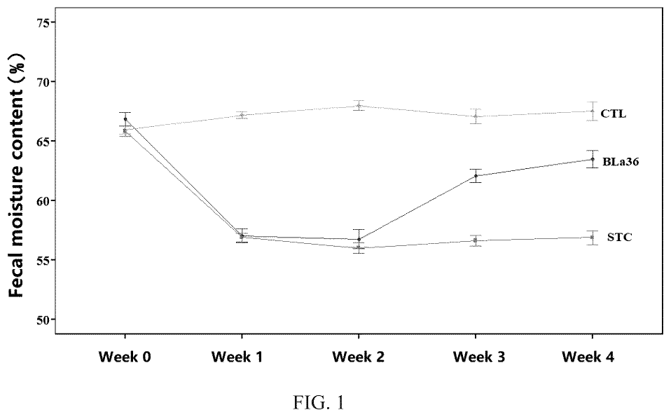

Animal feces were collected during the experiment. The wet weight of feces (A) was recorded immediately after collection and the dry weight of feces (B) was recorded after 3 hours of drying in a drying oven. The moisture content was calculated as follows: fecal moisture content (%)=(A−B)/A×100%. The results are shown in FIG. 1.

### (4) Evaluation of Intestinal Motility:

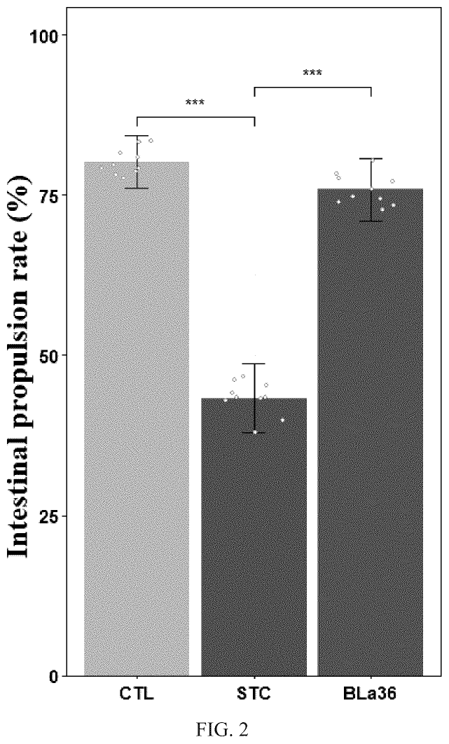

The intestinal transit rate (intestinal propulsion rate) was calculated as follows: intestinal transit rate (%)=C/D×100% (C: length between the pyloric sphincter and the end of the activated charcoal-stained intestines; D: full length of the intestines (the distance from pylorus to rectum). The results are shown in FIG. 2.

The results show that, as shown in FIG. 1, the fecal moisture content of normal mice (CTL group) is stable above 65%, while the fecal moisture content of slow transport constipation rats (STC group and BLa36 group) drops sharply within one week and then remains around 57%; mice in the BLa36 group are administrated with the Bifidobacterium animalis subsp. lactis BLa36 for treatment from the second week and its fecal moisture content is significantly increased, demonstrating the effect of Bifidobacterium animalis subsp. lactis BLa36 in restoring intestinal function. As can be seen in FIG. 2, the intestinal propulsion rate of the STC group is significantly lower than that of the CTL group (p<0.01), while the intestinal propulsion rate of the BLa36 group is higher than that of the STC group (p<0.01) and has approached the level of the normal group. Those results further indicate that the Bifidobacterium animalis subsp. lactis BLa36 has a significant effect on restoring intestinal function and can be applied to products for relieving or treating constipation.

### Example 6

In this example, the effect of Bifidobacterium animalis subsp. lactis BLa36 on neurotransmitters in slow transport constipation rats is explored, the steps of which are as follows.

(1) The constipated rat model was established in the same way as in Example 5.

### (2) Blood Sample Collection:

After 14 days of administration, rats were fasted for 12 h, but allowed to drink freely. Each rat was orally given 0.2 mL of standard charcoal meal, and after 30 minutes, anesthetized by intraperitoneal injection of sodium pentobarbital (50 mg/kg), and placed on a temperature-regulated table. Blood samples were collected and centrifuged at 3500 rpm for 15 min to obtain serum.

### (3) Blood Sample Analysis:

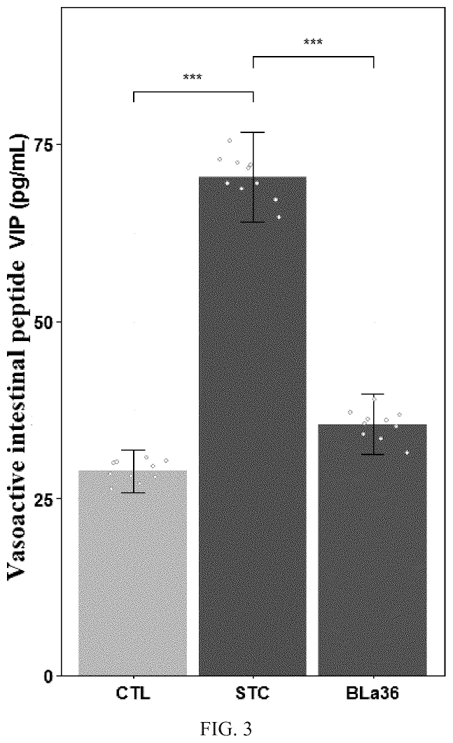

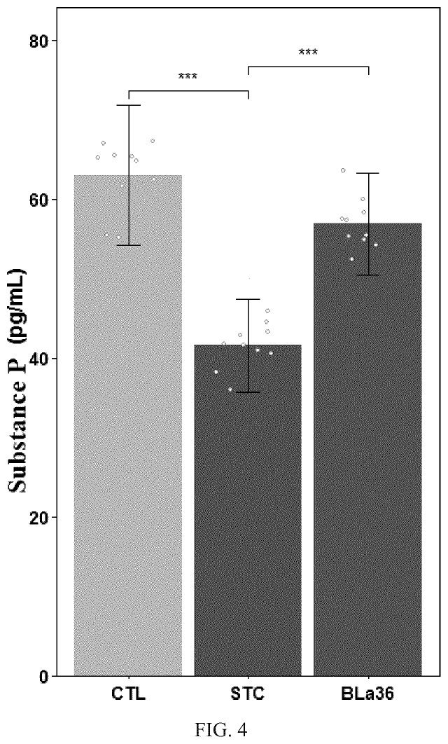

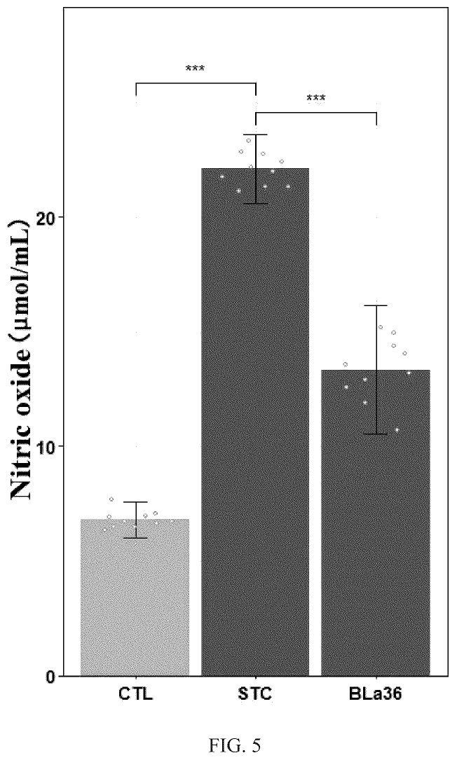

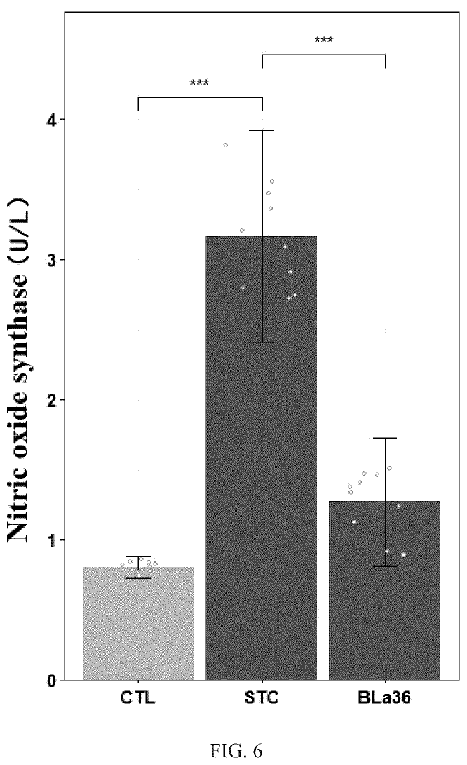

The serum was detected using commercial ELISA kits for concentrations of vasoactive intestinal peptide (VIP), substance P (SP), nitric oxide (NO) and nitric oxide synthase (NOS). The ELISA kits for VIP and substance P were purchased from Shanghai Fusheng Industrial Co., Ltd. The kits for Nitric oxide (NO) and nitric oxide synthase (NOS) were purchased from Nanjing Jiancheng Bioengineering Institute. The test methods were performed according to the protocols of the kits. The detection results of Bifidobacterium animalis subsp. lactis BLa36 intervention on vasoactive intestinal peptide (VIP), substance P (SP), nitric oxide (NO) and nitric oxide synthase (NOS) are shown in FIGS. 3, 4, 5 and 6, respectively.

The results show that compared with the normal rats (CTL group), rats in the STC group has significantly higher level of serum VIP (p<0.01), significantly lower level of substance P (p<0.01), and significantly higher levels of nitric oxide and nitric oxide synthase (p<0.01). After the administration with Bifidobacterium animalis subsp. lactis BLa36 for treatment, the serum VIP level of rats significantly decreases (p<0.01), the substance P level significantly increases (p<0.01), and the nitric oxide and nitric oxide synthase levels also significantly increase (p<0.01). The result indicates that the Bifidobacterium animalis subsp. lactis BLa36 enables the inhibitory neurotransmitters (VIP, NO, etc.) to significantly decrease and enables the substance P level to increase in the slow transport constipation rats.

### Example 7

In this example, the Bifidobacterium animalis subsp. lactis BLa36 is tested for the effect in improving ICC function, the steps of which are as follows.

(1) The constipated rat model was established in the same way as in Example 5.

### (2) Tissue Sample Collection:

After 14 days of administration, rats were fasted for 12 h, but allowed to drink freely. Each rat was orally given 0.2 mL of standard charcoal meal, and after 30 minutes, anesthetized by intraperitoneal injection of sodium pentobarbital (50 mg/kg), and placed on a temperature-regulated table. The colon was immediately harvested, rinsed with normal saline at 4° C., fixed in 10% formalin, and processed with paraffin for subsequent immunohistochemical studies.

### (3) Immunohistochemical Analysis:

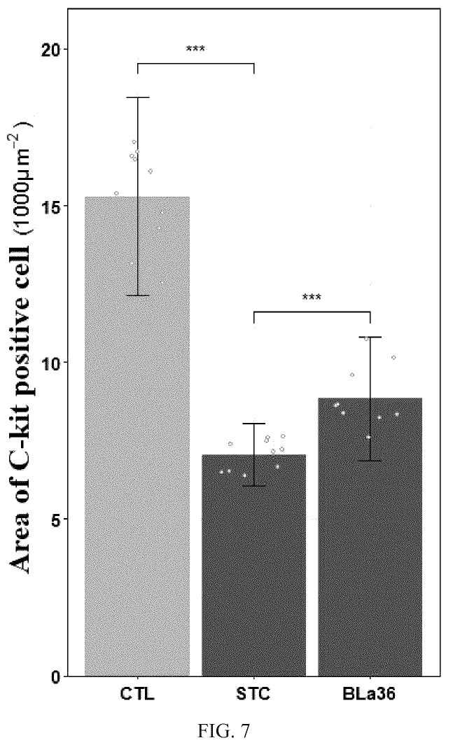

An unstained 5 μm section was cut from the paraffin block, and the section was stained with rabbit anti-c-Kit (purchased from Sigma-Aldrich) at 4° C. overnight (using the secondary antibody and avidin-biotin-peroxidase complex method). The nonspecific binding was corrected using an immunoglobulin negative control. The c-kit expression was determined by a semi-quantitative method. The results are shown in FIG. 7.

As can be seen in FIG. 7, the positive cell area of the STC group decreases compared with the CTL group, indicating that the constipated rats have reduced c-kit expression level in intestinal mucosa (p<0.01). The Bifidobacterium animalis subsp. lactis BLa36 treatment enables the reduced c-kit in constipated rats to significant increase (p<0.01).

### Example 8

In this example, the Bifidobacterium animalis subsp. lactis BLa36 is tested for the function to regulate intestinal flora, the steps of which are as follows.

(1) The constipated rat model was established in the same way as in Example 5.

### (2) Intestinal Flora Analysis:

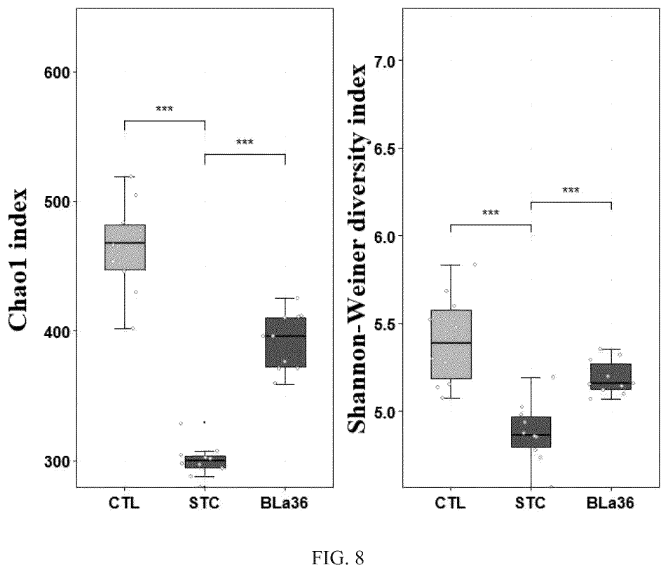

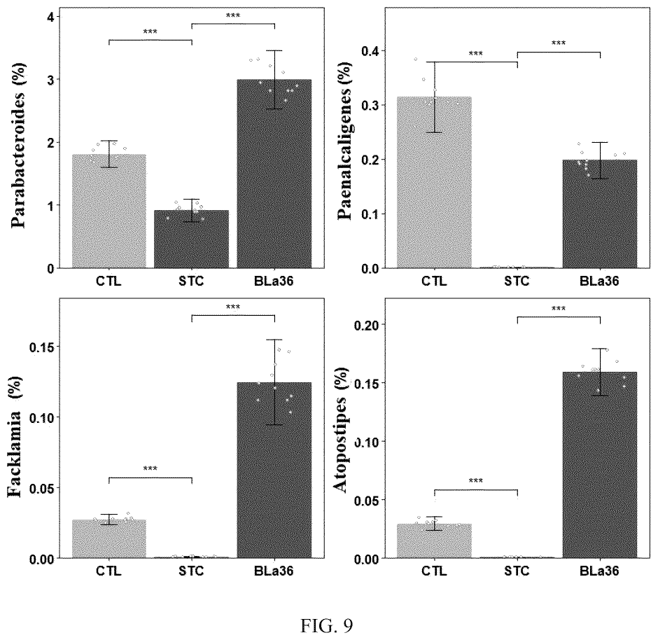

Rat feces were collected. Total DNA was extracted from 200 mg of feces using the QIAamp Fecal DNA Extraction Kit (Qiagen). The V3-V4 regions of 16S rRNA were amplified by PCR using 341F (SEQ ID No: 2: 5′-CCTACGGGNGGCWGCAG-3′) and 805R (SEQ ID No: 3: 5′-GACTACHVGGGTATCTAATCC-3′) as primers. The final 16S rRNA gene amplicon libraries were sequenced on MiSeq platform (Illumina) using 2×300 bp paired-end sequencing. The obtained paired-end reads were merged using Usearch11 and the reads with length≥400 pb were retained for the subsequent analysis. All the sequences after quality filtering were mapped to chimera-free ASV, and an ASV abundance table was created using USEARCH11 with default settings. The 16S rRNA database from the RDP training set (v18) was used as the reference database. Based on the ASVs abundance table, the Chao1 and Shannon-Weiner (Shannon-Weiner) diversity index (α-diversity) was calculated using Usearch and the intervention effects were determined by one-way analysis of variance (ANOVA), and differences between groups were analyzed by Tukey's post hoc test of significant difference. The intervention results of Bifidobacterium animalis subsp. lactis BLa36 on the α-diversity and differential flora are shown in FIG. 8 and FIG. 9, respectively.

As can be seen in FIG. 8, the α-diversity of the microbial community in the STC group significantly decreases compared with the normal group, including a decrease in microbial richness (reflected as Chao1 index) and a decrease in microbial diversity (reflected as Shannon-Weiner diversity index). As shown by the comparison of the BLa36 group with the STC group, the Bifidobacterium animalis subsp. lactis BLa36 increases the α-diversity of the microbial community. As shown in FIG. 9, compared with the STC group, the Bifidobacterium animalis subsp. lactis BLa36 significantly increases the relative abundance of Parabacteroides, Paenalcaligenes, Facklamia, and Atopostipes (ectopic eelworm, Carnobacterium).

### Example 9

This example provides a drug for relieving or treating constipation, which is prepared as follows.

The Bifidobacterium animalis subsp. lactis BLa36 was inoculated into the MRS liquid medium, incubated at 37° C. for 24 h for activation, and activated for 2 times consecutively to obtain an activation solution; the activation solution was inoculated into the MRS liquid medium at an inoculum amount of 2% (v/v), and incubated at 37° C. for 24 h to obtain a bacterial solution; the bacterial solution was centrifuged at 8000 g for 10 min to obtain the Bifidobacterium animalis subsp. lactis BLa36 bacteria;

the bacterial pellet were re-suspended with a skimmed milk powder having a mass concentration of 10% to a concentration of 1×10¹⁰ CFU/mL to obtain a bacterial suspension; the bacterial suspension was pre-incubated at 37° C. for 1 h and then lyophilized to obtain the drug for relieving or treating constipation.

### Example 10

This example provides a nutraceutical for relieving or treating constipation, and the nutraceutical for relieving or treating constipation comprises: 0.1 g of lyophilized powder of Bifidobacterium animalis subsp. lactis BLa36 for relieving or treating constipation and 1.9 g of inulin, which can be packed into sachets for consumption.

Packed as 2.0 grams per sachet, the product contains 2 billion CFU of probiotics per sachet. Inulin, a prebiotic, further improves the effectiveness of treating constipation when used in conjunction with the Bifidobacterium animalis subsp. lactis BLa36.

The lyophilized powder of Bifidobacterium animalis subsp. lactis for relieving or treating constipation is prepared as follows:

the Bifidobacterium animalis subsp. lactis BLa36 was inoculated into the MRS liquid medium at an inoculum amount of 3% of a total mass of the medium, and incubated at 37° C. for 18 h to obtain a culture solution; the culture solution was centrifuged at 8000 g for 10 min to obtain a bacterial pellet; the bacterial pellet was mixed with a skimmed milk powder solution which had a mass concentration of 10%, wherein a mass ratio of the skimmed milk powder to the bacterial pellet was 2:1, to obtain a re-suspension solution; the re-suspension solution was lyophilized by vacuum lyophilization method to obtaining the lyophilized powder of Bifidobacterium animalis subsp. lactis BLa36.

The establishment method of constipated rat model and the detection method of intestinal propulsion rate are the same as in Example 5.

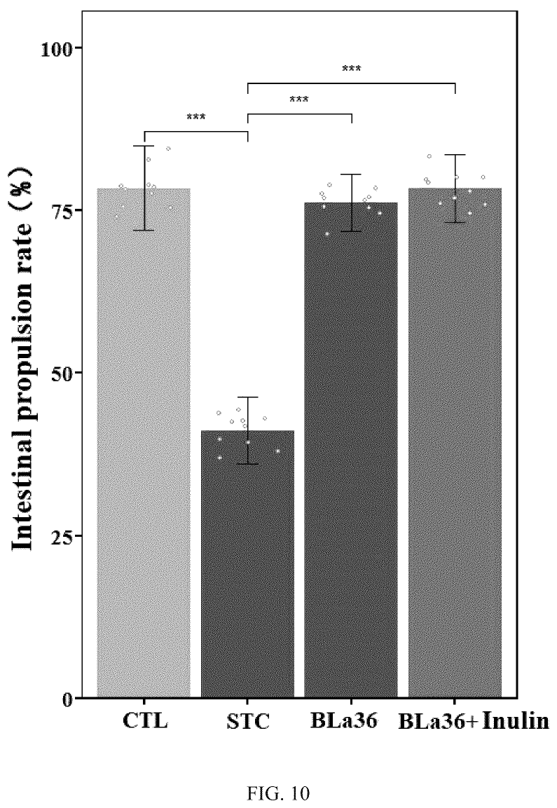

The nutraceutical for relieving or treating constipation was prepared into a re-suspension solution with water (in which the density of Bifidobacterium animalis subsp. lactis BLa36 was 10⁹ CFU/mL), and rats were gavaged to test the intestinal propulsion rate. The results are shown in FIG. 10.

As can be seen in FIG. 10, the intestinal propulsion rate of the STC group is significantly lower than that of the CTL group (p<0.01), while, after the treatment with the Bifidobacterium animalis subsp. lactis BLa36 or the nutraceutical prepared by compounding the Bifidobacterium animalis subsp. lactis BLa36 with inulin, the intestinal propulsion rate is higher than that of the STC group (p<0.01). The effect of intestinal function restoration is obvious and the bacteria can be applied to relieve constipation.

### Example 11

Comparison between the constipation-relieving effects of the Bifidobacterium animalis subsp. lactis BLa36 of the present application and other strains, and comparison among the constipation-relieving effects of composite probiotic preparations obtained by compounding various strains

In order to further demonstrate the excellent effect of the Bifidobacterium animalis subsp. lactis animalis BLa36 of the present application in relieving constipation, the standard strain of Bifidobacterium animalis subsp. lactis (ATCC27536) and Bifidobacterium animalis subsp. lactis animalis BLa80 were selected in the this example for comparative testing, and the test was carried out according to Example 5. Additionally, in this example, the above bacteria were compounded and subjected to comparative testing to investigate whether the constipation-relieving effect can be further improved by compounding two types of bacteria, the test was carried out according to Example 5. The administration dosage of each bacterial strain or each probiotic preparation combination was 10⁹ CFU (total count of bacteria)/day.

The results of fecal moisture content (average results) obtained for each bacterial strain or each probiotic preparation combination are shown in Table 2.

TABLE 2

| Group | Week 0 | Week 2 | Week 4 |
|---|---|---|---|
| CTL | 65.8% | 67.1% | 67.4% |
| STC | 67.4% | 55.7% | 56.8% |
| BLa36 | 67.5% | 57.0% | 63.7% |
| ATCC27536 | 66.1% | 56.3% | 59.3% |
| BLa80 | 67.3% | 56.2% | 58.8% |
| BLa36 + BLa80 (with a mass | 66.9% | 55.9% | 67.1% |
| ratio of 1:1) | | | |
| BLa36 + ATCC27536 (with a | 66.2% | 57.5% | 60.5% |
| mass ratio of 1:1) | | | |

The results show that compared with the standard strain of Bifidobacterium animalis subsp. lactis (ATCC27536) and Bifidobacterium animalis subsp. lactis BLa80, which have been disclosed in the prior art, the Bifidobacterium animalis subsp. lactis BLa36 of the present application has a significantly better effect in relieving constipation. In addition, it is also unexpectedly found in the present application that although the effect of BLa80 is slightly inferior to that of BLa36, the constipation-relieving effect of the composite probiotic preparation obtained by compounding BLa36 with BLa80 is surprisingly improved relative to BLa36 used alone. The two bacterial strains may promote each other's growth and thus have a synergistic effect in relieving constipation.

In conclusion, the present application provides Bifidobacterium animalis subsp. lactis BLa36 for relieving constipation, and the Bifidobacterium animalis subsp. lactis BLa36 has good gastric acid resistance and can relieve constipation; the corresponding product for relieving constipation, prepared from BLa36, will not cause any adverse reaction nor disrupt the balance of the intestinal flora, and at the same time, the corresponding product also avoids the constipation-related pathogens developing drug resistance, which is likely to be caused by the use of antibiotics, and has great application value.

The applicant declares that Bifidobacterium animalis subsp. lactis BLa36 of the present application for relieving constipation, a method for culturing the same and use thereof are illustrated in the present application through the examples, but the present application is not limited to the examples, which means that the present application is not necessarily relied on the examples for implementation. It should be clear to those skilled in the art that any improvement of the present application, equivalent substitution of raw materials of the product of the present application, addition of auxiliary ingredients, selection of specific methods, etc., shall fall within the protection and disclosure scope of the present application.

The preferred embodiments of the present application are described above in detail, however, the present application is not limited to the specific details of the embodiments, and within the scope of the technical conception of the present application, a plurality of simple variations may be carried out on the technical solutions of the present application, which shall all fall within the protection scope of the present application.

Additionally, it should be noted that the specific technical features described in the above embodiments can be combined in any suitable manner without contradiction, and in order to avoid unnecessary verbosity, the present application will not describe the various possible combinations.

1. Bifidobacterium animalis subsp. lactis BLa36 for relieving constipation, named as Bifidobacterium animalis subsp. lactis strain BLa36, which has been deposited in the China General Microbiological Culture Collection Center on Dec. 2, 2021 with a deposit number of CGMCC No. 24029, and a deposit address of No. 3, No. 1 Courtyard, West Beichen Road Chaoyang District Beijing.

2. A method for culturing the Bifidobacterium animalis subsp. lactis BLa36 for relieving constipation according to claim 1, comprising inoculating the Bifidobacterium animalis subsp. lactis BLa36 onto an MRS medium for culturing.

3. The method for culturing the Bifidobacterium animalis subsp. lactis BLa36 for relieving constipation according to claim 2, wherein the culturing is performed at 30-37° C. for 18-24 h.

4. A probiotic preparation for relieving constipation, comprising the Bifidobacterium animalis subsp. lactis BLa36 for relieving constipation according to claim 1 and a protective agent.

5. The probiotic preparation for relieving constipation according to claim 4, wherein the protective agent comprises a skimmed milk powder.

6. The probiotic preparation for relieving constipation according to claim 4, wherein the probiotic preparation for relieving constipation further comprises a prebiotic which comprises any one or a combination of at least two of oligofructose, oligogalactose, xylooligosaccharide, isomaltooligosaccharide, soybean oligosaccharide, inulin, spirulina, arthrospira, Coriolus versicolor polysaccharide, stachyose, polydextrose, α-lactalbumin or lactoferrin.

7. The probiotic preparation for relieving constipation according to claim 4, wherein the probiotic preparation for relieving constipation further comprises Bifidobacterium animalis subsp. lactis BLa80, named as Bifidobacterium animalis subsp. lactis strain BLa80, which has been deposited in the China General Microbiological Culture Collection Center on Mar. 5, 2018 with a deposit number of CGMCC NO. 15410, and a deposit address of No. 3, No. 1 Courtyard, West Beichen Road Chaoyang District Beijing.

8. The probiotic preparation for relieving constipation according to claim 7, wherein a mass ratio of the Bifidobacterium animalis subsp. lactis BLa36 to the Bifidobacterium animalis subsp. lactis BLa80 is (1-3):(1-3).

9. (canceled)

10. (canceled)

11. A method for relieving or treating constipation, comprising administering effective amount of a product containing the Bifidobacterium animalis subsp. lactis BLa36 for relieving constipation according to claim 1 to subject in need thereof;
wherein, the product comprises a foodstuff, a nutraceutical or a drug.

12. The method according to claim 11, wherein in the product, the Bifidobacterium animalis subsp. lactis BLa36 has a viable count of not less than 1×10⁸ CFU/mL.
